SEQUENCE LISTING (4 sequences)

SEQ ID NO: 1 - DNA, 48 bp, Artificial Sequence
  Sequence of primer for use with Cy3
ggccgactcactgcgcgtcttctgtcccgccttttttttttttttttt

SEQ ID NO: 2 - DNA, 48 bp, Artificial Sequence
  Sequence of primer for use with Cy5
cctgttgctctatttcccgtgccgctccggtttttttttttttttttt

SEQ ID NO: 3 - RNA, 12 nt, Eukaryotic Poly A Tail
  polyA_site (1)..(12)
aaaaaaaaaaaa

SEQ ID NO: 4 - DNA, 12 bp, Artificial Sequence
  Poly T for hybridization to Poly A tail
tttttttttttt

(19) United States
(12) Patent Application Publication
(10) Pub. No.: US 2005/0003366 A1
(43) Pub. Date: Jan. 6, 2005

(54) Method for reusing standard blots and microarrays utilizing DNA dendrimer technology

(76) Inventor: Robert C. Getts, Montvale, NJ (US)

Correspondence Address: LAW OFFICE OF MORRIS E. COHEN, 1122 CONEY ISLAND AVENUE, SUITE 217, BROOKLYN, NY, 11230, US

(21) Appl. No.: 10/643,596

(22) Filed: Aug. 19, 2003

Related U.S. Application Data
(63) Continuation of application No. PCT/US02/05022, filed on Feb. 20, 2002.
Application No. PCT/US02/05022 is a continuation of application No. 60/270,023, filed on Feb. 20, 2001, now abandoned.

(51) Int. Cl. C12Q 1/68; C12M 1/34

(52) U.S. Cl. 435/6; 435/287.2

(57) ABSTRACT

A method for reuse of standard blots and microarrays via removal of the capture reagent from the array, allowing multiple rounds of experiments using the same blot or microarray, without the need to remove the target molecules or the probe molecules attached to the support.

### RELATED APPLICATIONS

The present application is a continuation of PCT Application Serial No. PCT/US02/05022 filed 20 Feb. 2002, which claims the benefit of U.S. Provisional Patent Application Ser. No. 60/270,023 filed Feb. 20, 2001. The priority of both applications is hereby claimed, and both applications are fully incorporated herein by reference.

### FIELD OF THE INVENTION

The present invention is related generally to nucleic acid assays, more particularly to methods for reusing standard blots and microarrays.

### BACKGROUND OF THE INVENTION

Nucleic acid detection is traditionally performed by hybridizing two complementary strands of nucleic acid (DNA or RNA), one of which is the target and one of which is the probe, labeled nucleotides having been incorporated into one of the two strands to generate a detectable signal. The label may be a radioisotope such as ³²P, biotin, digoxigenin, various fluorescent molecules, or so forth, as is well known in the art.

One of the two nucleic acid strands is usually attached to some type of a support, such as a membrane (as with southern and northern blots), or such as a glass slide (as with microarrays). Usually a solid support is used, although other types of supports have also been disclosed in the art.

For microarrays, the nomenclature of the nucleic acid strands is generally such that the nucleic acid with known sequence affixed to the support is referred to as the “probe”, and the nucleic acid sequence to be detected in the sample is referred to as the “target”. However, this is not a universal nomenclature, since many in the art use a nomenclature wherein the meaning for probe and target are reversed. For blots, on the other hand, the nomenclature of the nucleic acid strands is generally such that the nucleic acid with known sequence affixed to the support is referred to as the “target”, and the nucleic acid sequence to be detected in the sample is referred to as the “probe”. There too, however, many in the art use a nomenclature wherein the meaning for probe and target are reversed.

As a result, for ease of reference, the terminology common for microarrays will be used hereinafter. As used in the present application for blots, the terms “target” known sequence” and “affixed molecules” (and similar variations thereof) are used to refer to the known nucleic acid sequences affixed to the assay solid support, and the terms “probe”, “unknown sequence”, “sample sequence”, and “sample molecules” (and similar variations thereof) are used to refer to the nucleic acids of the test sample whose identity is being investigated using the assay. However, it is to be understood that such nomenclature is merely provided for reference purposes and is not meant to be limiting.

Since the hybridization between the probe and target can be between nucleic acid sequences up to hundreds to thousands of base pairs long, the two hybridized strands are typically difficult to separate because of the high stability of the inter-strand hydrogen bonding. Thus, most blots and microarrays are difficult or impossible to reuse because the label is carried over from the first experiment to the next. As a result, a new blot or microarray must be prepared for each experiment that is conducted.

It would, therefore, be highly desirable to provide a blot or microarray which could be reused such that the known probe molecules affixed thereon could be utilized multiple times for a variety of different experiments. It would be a significant advance in gene expression detection microarrays to provide such a method, which would safely and efficiently provide a researcher or clinician with the ability to conduct a new experiment or test without the need to conduct a new attachment of the known probe molecules to a solid support.

It would further be desirable if such a method could be used with the emerging new technologies such as the recent microarray and dendrimer technologies. Developing DNA technologies provide rapid and cost-effective methods for identifying gene expression and genetic variations on a large-scale level. In particular, the DNA microarray is highly useful for rapidly detecting and assaying samples of target nucleic acid reagent. Each microarray is capable of performing the equivalent of thousands of individual “test tube” experiments over a short time period thereby providing rapid and simultaneous detection of thousands of expressed genes. Microarrays have been implemented in a range of applications such as analyzing a sample for the presence of gene variations or mutations (i.e. genotyping), or for patterns of gene expression.

Generally, a microarray comprises a substantially planar substrate such as a glass cover slide, a silicon plate or nylon membrane, coated with a grid of tiny spots or features of about 20 microns in diameter. Each spot or feature contains millions of copies of a specific sequence of nucleic acid extracted from a strand of deoxyribonucleic acid (DNA). Due to the number of features involved, a computer is typically used to keep track of each sequence located at each predetermined feature. Messenger RNA (mRNA) is extracted from a sample of cells. The mRNA, serving as a template, is reverse transcribed to yield a complementary DNA (cDNA). As a first example of the prior art techniques, one or more labels or markers such as fluorescence are directly incorporated into the copies of cDNA during the reverse transcription process. The labeled copies of cDNA are broken up into short fragments and washed over the microarray. Under suitable hybridization conditions, the labeled fragments are hybridized or coupled with complementary nucleic acid sequences (i.e. gene probes) attached to the features of the microarray for ready detection thereof. This labeling method has been commonly referred to as “direct incorporation”.

Upon hybridization of the cDNA to the microarray, a detectable signal (e.g. fluorescence) is emitted for a positive outcome from each feature containing a cDNA fragment hybridized with a complementary gene probe attached thereto. The detectable signal is visible to an appropriate sensor device or microscope, and may then be detected by the computer or user to generate a hybridization pattern. Since the nucleic acid sequence at each feature on the array (the probe) is known, any positive outcome (i.e. signal generation) at a particular feature indicates the presence of the complementary cDNA sequence in the sample cell. Although there are occasional mismatches, the attachment of millions of gene probes at each spot or feature ensures that the detectable signal is strongly emitted only if the complementary cDNA of the test sample is present.

Using known methods, a plurality of gene probes consisting of known nucleic acid sequences are each affixed or printed at a predetermined location on the surface of a microarray. The attachment of the gene probe to the microarray is typically accomplished through known robotic or laser lithographic processes.

The sample can be extracted from cells of organisms in the form of RNA. Since RNA is relatively unstable and decomposes rapidly and easily, a more stable and resistant form of nucleic acid is typically used. The stable nucleic acid is complementary DNA which is prepared from the RNA sample (e.g. total RNA and poly(A)+ RNA) through conventional techniques for implementing reverse transcription. Reverse transcriptase and reverse transcription primers (RT primers) having a capture sequence attached thereto, are used to initiate the reverse transcription process. This results in the formation of the target cDNA with the capture sequence located at the 5′ end. The newly formed target cDNA with the capture sequence is then isolated from the mRNA sample and precipitated. The target cDNA is hybridized to the complementary gene probes affixed to the microarray. After the target cDNA and the microarray are hybridized, the microarray is washed to remove any excess RT primers prior to labeling.

A mixture containing labeled “dendritic nucleic acid molecules”, or “dendrimers”, is then prepared. Dendrimers are complex, highly branched molecules, and are comprised of a plurality of interconnected natural or synthetic monomeric subunits of double-stranded DNA forming into stable spherical-like core structures with a predetermined number of “free ends” or “arms” extending therefrom. Dendrimers provide efficient means for labeling reactions such as fluorescence, for example, and facilitate direct calculations of the number of transcripts bound due to their predetermined signal generation intensity and proportional relationship to the bound cDNA on the microarray.

Each dendrimer includes two types of hybridization “free ends” or “arms” extending from the core surface. Each dendrimer may be configured to include at least one hundred arms of each type. The arms are each composed of a single-stranded DNA of a specific sequence that can be ligated or hybridized to a functional molecule, such as a target molecule or a label. The dendrimer in conjunction with the target molecule has the capability to target and hybridize to a complementary sequence of probe affixed to the array. The label molecule can be attached to the other type of arm to provide the dendrimer with signal emission capabilities for detection of the dendrimer, signalling a hybridization even thereof. The dendrimer is typically hybrized to the target molecule by providing a nucleotide sequence on an arm of the dendrimer that is complementary to the capture sequence of the target molecule, and the label molecule is typically an oligonucleotide linked to a label or marker. Using simple DNA labeling, hybridization, and ligation reactions, a dendrimer can thus be configured to act as a highly labeled, target-specific molecule, and therefore may be used in a microarray system for DNA analysis. Dendrimer technology is described in greater detail in U.S. Pat. Nos. 5,175,270 and 5,484,904, in Nilsen et al., Dendritic Nucleic Acid Structures, J. Theor. Biol., 187, 273-284 (1997); in Stears et al., A Novel, Sensitive Detection System for High-Density Microarrays Using Dendrimer Technology, Physiol. Genomics, 3:93-99 (2000); prior patent applications by the present inventor, such as PCT application Ser. No. PCT/US01/07477; and published protocols available from Genisphere, Inc. of Montvale, N.J.; all of which are fully incorporated herein by reference.

The prepared mixture is formulated in the presence of a suitable buffer to yield a dendrimer hybridization mixture containing dendrimers with labels attached to one type of arm, and with oligonucleotides complementary to the capture sequences of the target cDNA attached to the other type of arm. The labeled dendrimers are added to the microarray for hybridization of the capture sequence complement of the dendrimer with the capture sequences of the bound cDNA probe to generate a detectable signal from the corresponding feature. The microarray is washed to remove any excess unhybridized dendrimer molecules to reduce unwanted noise generation. The microarray is scanned using conventional techniques to detect the signal emitted by the labels to generate a particular hybridization pattern for analysis. Signal detection indicates the presence of hybridization of molecules in the sample to a feature (a probe) on the microarray. Since the probes affixed to the each position on the microarray are of known sequence, the signal provides important sequence information about the previously unknown sequences of the sample.

However, in the traditional methods of the prior art, an assay can only be used once. A new assay, with probe molecules thereon for the bound cDNA target to bind to, must be prepared for each new experiment.

### SUMMARY OF THE INVENTION

It is an object of the present invention to provide a method for reusing standard blots and microarrays.

It is a further object of the present invention to provide a method for reusing standard blots and microarrays using capture reagents.

It is a further object of the present invention to provide a method for use and reuse of standard blots and microarrays by efficient removing the capture reagents from target molecules of a prior experiment that have hybridized to probes affixed to the solid support.

It is a further object of the present invention to provide a method for reusing standard blots and microarrays using dendrimer technology.

Further objects and advantages of the invention will become apparent in conjunction with the detailed disclosure provided herein.

In accordance with the present invention, a method is provided allowing the reuse of standard blots and microarrays. In the past, reuse has been extremely difficult or impossible, due to the fact that the hybridization between probe and target is traditionally between nucleic acid sequences up to hundreds to thousands of base pairs long. The considerable length of hybridized sequence results in conditions strongly disfavoring separation, because of the high stability of the inter-strand hydrogen bonding. As a result of the inability to effectively separate the strands, most blots and microarrays cannot be used again in a subsequent experiment, since the label would be carried over from the first experiment to the next.

In contrast, the method of the present invention provides for reuse by removal of the capture reagent from the array, allowing multiple rounds of experiments using the same blot or microarray, without the need to remove the target molecules (or the probe molecules attached to the support). In accordance with the present invention, separation is performed at the binding site between the capture reagent and the target. Preferably, a short sequence of nucleic acid is separated binding the capture reagent to the target, allowing removal of the capture reagent with much greater ease than separation of the target from the probe. Further preferably, separation is conducted of a 31 nucleotide base pair hybrid between a capture sequence located on the probe or target and the complementary sequence attached to a capture reagent. As a result, a superior method for stripping and reusing the blot or array is provided.

In the preferred embodiment, the capture reagent is a dendrimer. Further preferably, a DNA dendrimer is used. DNA dendrimer technology has previously been described, for example, in US. Pat. Nos. 5,175,270; 5,484,904; 5,487,973; 6,072,043; 6,110,687; and 6,117,631; all of which are fully incorporated herein by reference. As disclosed therein, a nucleic acid target is detected by adding a binding site known as a “capture sequence” to the end of one of the two single strands in a nucleic acid hybridization assay (or by using an existing sequence), and hybridizing the capture sequence to a complementary sequence on a signal-carrying dendritic molecule. The capture sequence is unique to the probe or target nucleic acid sequence depending on the assay format, blot or microarray respectively.

Pursuant to the preferred method disclosed herein, the invention utilizes two or more unique capture sequences (and their corresponding complements). The method of reuse includes four steps or sets of steps: (1) initial hybridization of a first sample; (2) stripping; (3) detection; and (4) rehybridization using a second sample.

The method can be used with any desired assay format, whether blot, microarray, or so forth. Although reference will generally be made to arrays for illustration purposes, it is to be understood that the invention is not limited to arrays, but may be used with blots or any other assay formats currently in use or later developed in the art. Similarly, while the use of DNA dendrimers constitutes the preferred embodiment, other capture reagents currently in use or later developed can be used as well, consistent with the invention. For example, the present invention can be used with antibody-antigen conjugates, or other biomolecules which can be functionally or chemically designed to have appropriate binding capabilities, such as derivatized proteins, lipids, or so forth, whether conjugated to a nucleic acid or not. For purposes of illustration, the method will be described with respect to the preferred embodiment, although other capture reagents can be substituted consistent with the invention.

The first step, the initial hybridization of sample to the assay format, is a first experiment using capture reagent technology as known in the art. This experiment involves hybridization of a first set of target molecules of unknown sequence to the probe molecules of known sequence affixed to the format, and hybridization of a first set of capture reagents to those target molecules. The target molecules of this initial step have a capture sequence thereon, and the capture reagents (preferably dendrimers) have a complementary sequence to that capture sequence, so that the dendrimers and target molecules will hybridize. The capture sequence used for the target molecules of this first experiment are referred to herein as “the first capture sequence” or capture sequence A, and the complementary sequence on the arms of the dendrimers are likewise referred to as “the first capture sequence complement” or capture sequence A′.

Hybridization of one or more types of targets can be conducted to the array, e.g., using single or dual channel detection, as known in the art. For each type of target in the , a different capture sequence is used (capture sequences A1 and A2). For ease of illustration, the present example shall continue by reference to single channel detection.

After hybridization, standard detection of the signal from these DNA dendrimers can be performed, to complete the first experiment.

The second step, a “stripping step”, is performed to remove all bound and labeled dendrimer from the assay, so as to prepare the assay for reuse. Although the first set of dendrimers are removed, the probe molecules are left attached to the assay format. Similarly, the target molecules are left hybridized on the array to their complementary probes.

In the third step, a signal detection is conducted to confirm the prior removal of the label. Stripping should have removed all labelled dendrimer from the assay; in the event that any label remains, steps two and three can be repeated.

In the fourth step, the rehybridization step, a new experiment is conducted using the same assay format, but a new sample. In this second experiment, a second set of target molecules and a second set of dendrimers are used. The targets and dendrimers of this fourth step utilize a second capture sequence (B) and second capture sequence complement (B′) that are unique from those of the first step (A and A′ respectively). In other words, a capture sequence is used for the second experiment which is different from the capture sequences of the first experiment.

Since the second dendrimer-target hybridization (of step four) is performed using a different capture sequence than the first experiment (step one), only the dendritic reagent must be stripped between the two experiments, and not the difficult to remove full length target molecule. Furthermore, left-over capture sequence from a target molecule of the prior experiment can not bind to any dendrimers in the second experiment, since the dendrimers of the second experiment are designed to bind to a different capture sequence. As a result, the signal emitted by the dendrimers of this second experiment is not affected by the results of the first experiment.

This process can be repeated for as many cycles as desired, merely by using additional capture sequences (with complementary capture sequence oligonucleotide pairs). A new capture sequence is used for each subsequent experiment, the capture sequence being different from the capture sequences of all prior experiments. For example, the process can be continued with a third dendrimer-probe hybridization using a third capture sequence (C) (and complement) that is different from both the first and second capture sequences (A and B), and so on.

Accordingly, in one embodiment of the invention, a method is provided, comprising the steps of stripping a first label from a first target nucleic acid hybridized to a probe nucleic acid on an assay format; and, reusing the assay format by hybridizing a second target nucleic acid to probe nucleic acid on the assay format, the second target nucleic acid comprising a second label distinct from the first label.

In a further embodiment, a method is provided comprising the steps of stripping a first capture reagent from a first target nucleic acid hybridized to a probe nucleic acid on an assay format, wherein the first target nucleic acid initially comprises a first capture sequence of nucleic acid hybridized to complementary nucleic acid of the first capture reagent, and the stripping comprises separation of said hybridized first capture sequence of nucleic acid and complementary nucleic acid of first capture reagent. Preferably, the capture reagent is a dendrimer.

In a further embodiment, a method is provided comprising the steps of:
(a) conducting a first assay, the first assay comprising:
   (i) a first hybridization of a target nucleic acid to probe nucleic acid located on an assay format, and
   (ii) hybridization of a first capture reagent to said target nucleic acid, wherein the target nucleic acid comprises a first capture sequence which hybridizes with a complementary nucleic acid sequence of the first capture reagent;
(b) stripping the first capture reagent from the target nucleic acid; and,
(c) conducting a second assay on the assay format, the second assay comprising:
   (i) a second hybridization of target nucleic acid to probe nucleic acid on the same assay format used for the first assay; and,
   (ii) hybridization of a second capture reagent to the target nucleic acid of said second assay, wherein the target nucleic acid of the second assay comprises a second capture sequence for hybridization to the second capture reagent, the second capture sequence being a nucleic acid sequence which is different from the nucleic acid sequence of the first capture sequence.

In a further preferred embodiment, a method is provided comprising the steps of:
(a) conducting a first assay, the first assay comprising:
   (i) a first hybridization of a target nucleic acid to probe nucleic acid located on an assay format, and
   (ii) hybridization of a first dendrimer to the target nucleic acid, wherein the target nucleic acid comprises a first capture sequence which hybridizes with a complementary nucleic acid sequence of the first dendrimer;
(b) stripping the first dendrimer from said target nucleic acid; and,
(c) conducting a second assay on the assay format, the second assay comprising:
   (i) a second hybridization of target nucleic acid to probe nucleic acid on the same assay format used for the first assay; and,
   (ii) hybridization of a second dendrimer to the target nucleic acid of the second assay, wherein the target nucleic acid of said second assay comprises a second capture sequence for hybridization to the second dendrimer, the second capture sequence being a nucleic acid sequence which is different from the nucleic acid sequence of the first capture sequence.

Preferably, a detection step is further provided after each stripping step, to ensure that no label remains which will interfere with the results of a subsequent experiment.

### BRIEF DESCRIPTION OF THE DRAWINGS

FIG. 1 is a schematic representation of the preparation of a microarray or blot for detection and assay of a nucleic acid sequence sample using single channel analysis, in accordance with a first step of an embodiment of the present invention.

FIG. 2 is a schematic representation of the stripping of labelled dendrimer off of the microarray or blot of FIG. 1, in accordance with a second step of the embodiment of FIG. 1.

FIG. 3 is a schematic representation of the reuse of the microarray or blot of FIG. 1 in a new single channel assay, using a capture sequence distinct from that used in the assay of FIG. 1.

FIG. 4 is a schematic representation of the preparation of a microarray or blot for detection and assay of a nucleic acid sequence sample, in accordance with a first step of an alternative embodiment of the present invention using dual channel analysis.

FIG. 5 is a schematic representation of the stripping of labelled dendrimer off of the microarray or blot, in accordance with a second step of the embodiment of FIG. 4.

FIG. 6 is a schematic representation of the reuse of the microarray or blot of FIG. 4 in a new dual channel assay, using two new capture sequences distinct from those used in the assay of FIG. 4.

FIG. 7 is a schematic representation of a process for the creation of a target nucleic acid using a capture sequence, in one embodiment of the present invention.

FIG. 8 is a schematic representation of a process for microarray detection for use, for [symbol] example, in RNA expression analysis, in conjunction with the present invention.

### DETAILED DESCRIPTION OF THE INVENTION AND THE PREFERRED EMBODIMENTS

The present invention is generally directed to a method for conducting an analysis on an [symbol] assay format (e.g. a blot or microarray) in such a manner which significantly reduces the time effort typically required for preparing the assay. The method of the present invention [symbol] provides the advantage of allowing the reuse of the blot or microarray having probe nucleic acid [symbol] mixed thereto, thereby allowing a series of sequential experiments to be conducted on a single or microarray using new samples. The invention, therefore, provides a significant [symbol] advantage over the prior art which requires preparation of a new blot or microarray for each [symbol] criment.

The invention is suitable for both laboratory and clinical use. The cost effective and [symbol] efficient manner by which the target nucleic acid reagent is prepared and by which the method of [symbol] present invention can be implemented using conventional laboratory techniques, equipment reagents, makes the present invention especially suitable for use in genomic applications as gene expression profiling and high-throughput functional genomic analysis. The term [symbol] “target nucleic acid reagent” and “probe nucleic acid” as used herein are meant to encompass [symbol] DNA or RNA-based genetic material processed or extracted for assay on a blot, microarray, [symbol] her format.

Before the present invention is further described, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In accordance with the present invention, a first set of one or more target nucleic acids and one or more capture reagents (preferably in the form of a dendrimer), are concurrently contacted with an assay format, such as a microarray comprising a plurality of gene probes or a blot. The assay format is then treated to allow reuse of the format using a new set of target nucleic acid(s) and capture reagent(s).

This concurrent contact may be made individually with each reagent being applied to a microarray or blot relatively simultaneously, and then allowing the components to mix on the assay format. Or, in an alternate embodiment, the target nucleic acid and the capture reagent, preferably in the form of a dendrimer, are mixed to yield a mixture. This mixture is then contacted with the microarray comprising a plurality of gene probes or the blot. The hybridization between the target nucleic acid reagent and the microarray (or blot), and between the target nucleic acid reagent and the capture reagent (e.g. dendrimer) may be carried out in any suitable order. The capture reagent (e.g. dendrimer) is labeled and thus capable of generating the same signal of known intensity, thus each positive signal in the microarray can be “counted” in order to obtain quantitative information about the genetic profile of the target nucleic acid reagent.

The target nucleic acids can be provided from any suitable source, whether synthesized, derived from a biological sample, or so forth. The assay format is treated at a temperature and for a time sufficient to induce hybridization between the target nucleic acid reagent and the complementary gene probes of the blot or microarray, and thereafter induce the capture reagent to hybridize with the target nucleic acid reagent, whereupon a detectable signal may be generated to render the particular hybridization pattern visible.

In one embodiment of the methods of the present invention, the assay format is an array of DNA or gene probes fixed or stably associated with the surface of a substrate (normally substantially planar) is prepared as conventionally known in the art. A variety of different microarrays that may be used as is well known. The substrates with which the gene probes are stably associated may be fabricated from a variety of materials, including plastic, ceramic, metal, gel, membrane, glass, or so forth. The microarrays may be produced according to any convenient methodology, such as pre-forming the gene probes and then stably associating them with the surface of the support or growing the gene probes directly on the support. A number of different microarray configurations and methods for their production are known to those of skill in the art, as described, for example, in Science, 283, 83, 1999, the content of which is fully incorporated herein by reference.

In an alternate embodiment, the assay format is a classical blot assay. For this type of assay, cellular nucleic acid DNA or RNA is separated by size on an agarose gel and is subsequently transferred (blotted) to a solid support, known as a membrane. Such blots can be prepared by methods familiar to those skilled the art.

The nucleic acids of the gene probes of the microarrays or blot and the target nucleic acid reagent are capable of sequence specific hybridization, and may each be comprised of polynucleotides or hybridizing analogues or mimetics thereof, including, but not limited to, nucleic acid in which the phosphodiester linkage has been replaced with a substitute linkage group, such as phosphorothioate, methylimino, methylphosphonate, phosphoramidate, guanidine and the like, nucleic acid in which the ribose subunit has been substituted, e.g. hexose phosphodiester; peptide nucleic acid, or so forth. The length of the gene probes will generally range from 10 to 1000 nucleotides. In the preferred embodiment, the DNA or gene probes are each arranged or sequenced for hybridization with the target nucleic acid reagent, e.g. cDNA from a gene of concern.

In some embodiments of the invention, the gene probes will be oligonucleotides having from 15 to 150 nucleotides and more usually from 15 to 100 nucleotides. In other embodiments the gene probes will be longer, usually ranging in length from 150 to 1000 nucleotides (or longer), where the polynucleotide probes may be single or double stranded, usually single stranded, and may be PCR fragments amplified from cDNA, cloned genes, or other suitable sources of nucleic acid sequences. The DNA or gene probes on the surface of the substrates will preferably correspond to, but are not limited to, known genes of the physiological source being analyzed and be positioned on the microarray at a known location so that positive hybridization events may be correlated to expression of a particular gene in the physiological source from which the target nucleic acid reagent is derived. If the target nucleic acid reagent is generated in the form of DNA, as herein described below, the microarrays of gene probes will generally have sequences that are complementary to the DNA-based strands, including but not limited to, cDNA strands, of the gene to which they correspond.

The term “label” is used herein in a broad sense to refer to agents that are capable of providing a detectable signal, either directly or through interaction with one or more additional members of a signal producing system. Labels that are directly detectable and may find use in the present invention include but are not limited to, for example, alkaline phosphatase, biotin, digoxigenin, fluorescent labels such as fluorescein, rhodamine, BODIPY, cyanine dyes (e.g. from Amersham Pharmacia), Alexa dyes (e.g. from Molecular Probes, Inc.), fluorescent dye phosphoramidites, and the like; and radioactive isotopes, such as 32 S, 32 P, 3 H, etc.; or so forth. Examples of labels that provide a detectable signal through interaction with one or more additional members of a signal producing system include capture moieties that specifically bind to complementary binding pair members, where the complementary binding pair members comprise a directly detectable label moiety, such as a fluorescent moiety as described above. The label is one that preferably does not provide a variable signal, but instead provides a constant and reproducible signal over a given period of time.

The present invention further utilizes a capture reagent which is composed of at least one first arm containing a label capable of emitting a detectable signal and at least one second arm having a nucleotide sequence complementary to a capture sequence attached to the target nucleic acid such as DNA, for example. One such example is a “dendritic nucleic acid molecule”, or “dendrimer”. Briefly, dendrimers are complex, highly branched molecules, and are comprised of a plurality of interconnected natural or synthetic monomeric subunits of double-stranded DNA forming into stable, spherical-like core structures with a pre-determined number of “arms” or “free ends” extending therefrom for the purposes described herein. Typically, the capture reagent will have multiple, typically many, first and second arms. Besides dendrimers, carbohydrates, proteins, nucleic acids, and the like may be used as the capture reagent. The use of DNA dendrimers constitutes the preferred embodiment; however, other capture reagents currently in use or later developed can be used as well, consistent with the invention. For example, the present invention can be used with antibody-antigen conjugates, or other biomolecules which can be functionally or chemically designed to have appropriate binding capabilities, such as derivatized proteins, lipids, or so forth, whether conjugated to a nucleic acid or not. DNA dendrimers will be described hereinafter as illustrative of suitable capture reagents.

Each dendrimer may be configured to include two types of hybridization “free ends” or “arms” extending from the core surface. Each dendrimer may be configured to include at least one hundred arms of each type. The arms are each composed of a single-stranded DNA of a specific sequence that can be ligated or hybridized to a functional molecule such as a target or a label. The target molecule can be attached to one type of arm to provide the dendrimer with targeting capabilities, and the label molecule can be attached to the other type of arm to provide the dendrimer with signal generation capabilities for detection. The targeting molecule is typically an oligonucleotide that is complementary to the capture sequence of the target nucleic acid reagent, and the label molecule is typically an oligonucleotide linked to a label or marker. Using simple DNA labeling, hybridization, and ligation reactions, a dendrimer may be configured to act as a highly labeled, target specific binding molecule, and therefore may be used in a microarray system for DNA analysis.

For example, fluorescent labeled dendrimers may be prepared by ligating a nucleic acid sequence or strand complementary to the capture sequence of a target nucleic acid reagent to the purified dendritic core material as prepared by previously described methods (see Nilson et al., and Stears et al., supra; and the '270, '904, and '973 patent citations as previously mentioned). Labeled dendrimers ligated with the capture sequence are able to target and hybridize with a target nucleic acid reagent with a specific capture sequence attached thereto.

A dendrimer commonly used in the art may be obtained from the product 3DNA™ expression array reagent which is available from Genisphere Inc. and Datascope Corp. of Montvale, N.J. The application of the 3DNA™ reagent, is relatively straightforward. 3DNA™ reagent is available with either Cy3™ or Cy5™ labels attached thereto, making possible either single or dual channel detection in microarray assays. The labeled 3DNA™ capture reagent further may be adapted to include a “capture sequence complement” i.e. a nucleotide sequence that that is complementary to the 5′ end of a RT primer used to produce the target nucleic acid reagent which enables the capture reagent to hybridize to target nucleic acid reagent under suitable conditions during assay.

The labeled 3DNA™ capture reagent provides a more intense, predictable and consistent signal than the direct incorporation method described above, for two reasons. First, since the fluorescent dye is part of the 3DNA™ capture reagent, it does not have to be incorporated during the preparation of the target nucleic acid reagent (e.g., cDNA), thus avoiding the inefficient and unpredictable enzymatic incorporation of fluorescent dye nucleotide conjugates into the reverse transcript. Second, because each 3DNA™ capture reagent contains an average of about 250 or more fluorescent dyes and each target nucleic acid hybridized to the microarray can be readily detected by a single 3DNA™ capture reagent, the signal generated from each message will be largely independent of base composition or length of the corresponding transcript.

Further information regarding the structure, configuration and production of dendrimers is also disclosed in U.S. Pat. Nos. 5,175,270, 5,484,904, and 5,487,973, all of which are fully incorporated herein by reference. Furthermore, the present invention can be conducted in conjunction with any of the other inventions, methods and techniques provided by the present inventor, or by Genisphere, Inc. or Datascope, Inc. of Montvale, N.J.

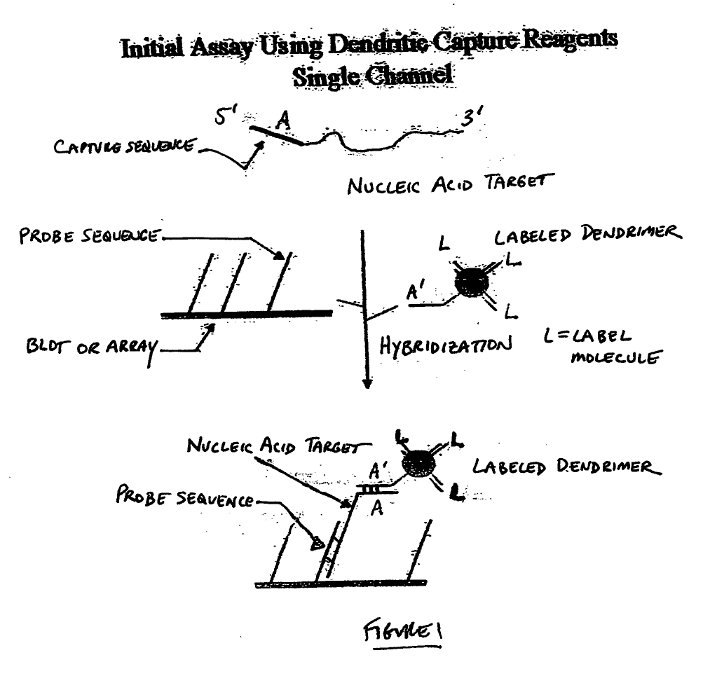

An illustration of the first step of a assay in accordance with one embodiment of the present invention (“step one” of the invention), whether using a microarray or a blot is shown in FIG. 1. (The figure illustrates a single channel hybridization; a dual channel hybridization can likewise be conducted, as discussed below).

In accordance with this first step of invention, an assay format is obtained or prepared, along with a target nucleic acid sample for analysis. The assay format has probe nucleic acid thereon, the assay format, for example, being in the form of a microarray or a blot. The target nucleic acid sample is treated for incorporation of a capture sequence within it, i.e. an additional sequence designed for the purpose of binding the target nucleic acid to a capture reagent.

A capture reagent, preferably a dendrimer, coupled to an oligonucleotide complementary to the capture sequence of the target nucleic acid reagent (“capture sequence complement”), is added to the target nucleic acid reagent to yield a hybridization mixture. The capture sequences and the complementary oligonucleotide have sufficient base units to hybridize under suitable conditions including time and temperature sufficient for promoting the hybridization of the dendrimer to the target nucleic acid reagent as known by those of ordinary skill in the art. In accordance with a preferred embodiment of the present invention, the capture sequence (and its complement attached to the dendrimer) are each 31 bases in length to form a 31 base pair hybrid. Suitable hybridization conditions are disclosed in Maniatis et al., where conditions may be modulated to achieve a desired specificity in hybridization. It is further noted that any suitable hybridization buffers may be used in the present invention.

The components (i.e., capture reagent and target nucleic acid reagent) of the hybridization mixture are then contacted with a microarray or blot comprising multiple features each containing a specific nucleic acid sequence (typically in the form of a fragment of a cDNA, although any source for the nucleic acid sequences may be utilized). As noted, the method of the present invention also encompasses applying the capture reagent and the target nucleic acid reagent (cDNA) to the microarray to yield the hybridization mixture upon contact.

Alternately, the components can be used in a classical blot assay. For this type of assay, cellular nucleic acid DNA or RNA is separated by size on an agarose gel and is subsequently transferred (blotted) to a solid support, known as a membrane, by methods familiar to those skilled the art. The nucleic acid is typically referred to as the target. A typical blot hybridization assay is conducted using a blot of the combined target molecules and dendritic DNA. Oligonucleotides labeled with alkaline phosphatase, biotin, digoxigenin or 32P or other label are added during the hybridization. The labeled oligonucleotides bind to the dendritic regent thus delivering signal.

Whether a microarray or blot is used, the assay format is treated at a temperature and for a time sufficient to induce hybridization between the target nucleic acid reagent and the complementary gene probes, and thereafter induce the capture reagent to hybridize with the target nucleic acid reagent, whereupon a detectable signal may be generated to render the particular hybridization pattern visible.

At this point, an initial hybridization has taken place in the same manner as the hybridizations typically conducted in the art using dendritic reagents. However, unlike typical hybridizations, the blot or microarray can now be reused, as follows.

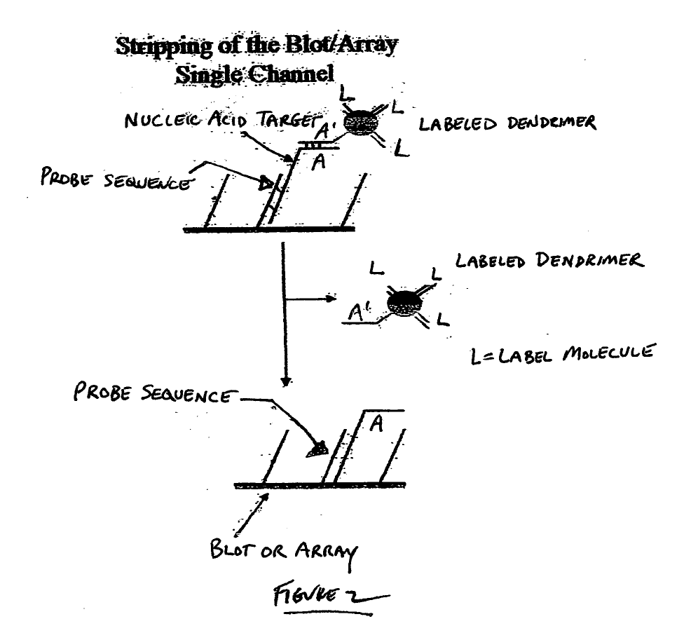

In a second step of the invention, all of the labelled dendrimers of step one are removed or “stripped” from the microarray or blot, as illustrated in FIG. 2. The microarray or blot is treated under suitable conditions to disrupt the hybridization between the capture sequence (A) of the target molecule and the capture sequence complement (A′) ligated to the arm of the dendrimer. Separation of this dual stranded nucleic acid strand causes the dendrimer to be released. For example, the microarray or blot can be washed in 0.2% SDS at 80° C. until the labelled dendrimer has been completely removed as determined by the standard detection procedure for the label used. Of course, this step is not limited to the use of the 0.2% SDS solution or temperature described, those conditions merely being provided as an illustrative embodiment. Any suitable stripping protocol can be employed.

Preferably, a 31 base pair hybrid is provided in step one to facilitate the separation in step two. The provision and subsequent disruption of this short hybridized sequence makes it far easier to “clean” the blot than attempting to separate the hundreds or thousands of base pairs of hybridization between the target molecule and the probe. Of course, the capture sequence/capture sequence complement does not need to be 31 base pairs in length, as other lengths can be utilized. However, this length provides a suitable balance in that the hybrid is sufficiently long to provide the stability desired for the process of step one, yet sufficiently short to allow the disruption needed for the process of step two.

In step three of the invention, the microarray or blot is scanned to detect any label thereon, using the standard procedure for the specific label used, as per procedures familiar to those skilled in the art. At this point, no labelled dendrimer should be detected as a result of the stripping of step two. However, should any label remain, the user can rewash the microarray or blot until all labelled dendrimer is gone.

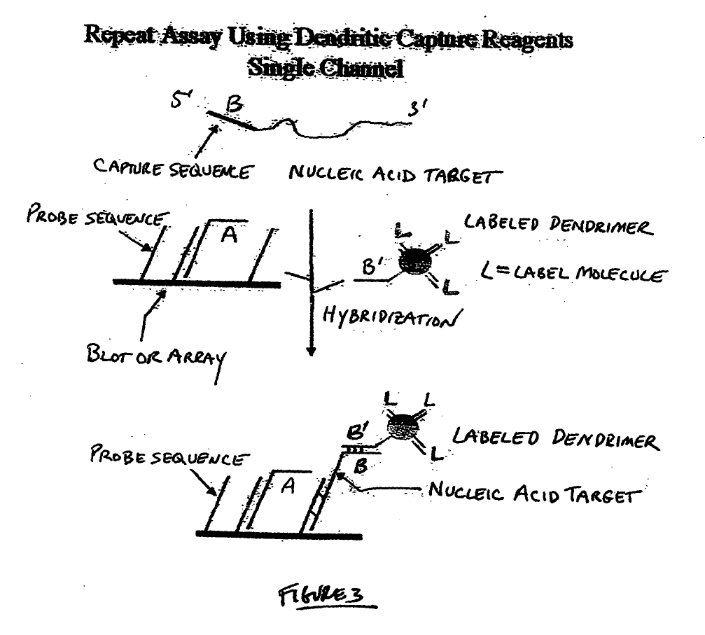

In step four of the invention, a new assay is conducted as shown in FIG. 3. In this second experiment, a second set of target molecules and a second set of dendrimers are used. This second experiment is prepared and conducted in similar fashion to the previous experiment of step one. However, in contrast to prior art methods, this new assay can be conducted on the same microarray or blot surface that was used for the first experiment. This is due to the fact that the assay now uses a new capture sequence (sequence B) and its complement (B′) on the dendrimer, as opposed to the experiment of FIG. 1, which used capture sequence A.

Capture sequence B is a distinct nucleic acid sequence from the nucleic acid of prior capture sequence A, such that the dendrimers of the second experiment having complement B′ attached thereto, cannot bind to the target molecules of the first experiment having capture sequence A attached thereto. These new dendrimers with complement B′ can only bind to the desired target molecules which have capture sequence B. Since the second dendrimer-target hybridization (that of step four) is performed using a different capture sequence than was used during the first experiment (step one), only the dendritic reagent need be stripped between the two experiments. The difficult to remove full length target molecule is left hybridized to the probe molecules. Any left-over capture sequences (A) from the target molecule of the prior experiment do not generate a signal, since the second set of dendrimers can not bind to them, and, thus, they do not affect the second assay's results.

This process can be repeated as often as desired, merely by using a new capture sequences (with complementary capture sequence oligonucleotide pairs) for each new cycle. The capture sequence of each new cycle (each new assay) is different from the capture sequences of all prior cycles. For example, the process can be continued with a third dendrimer-probe hybridization using a third capture sequence (C) (and complement) that is different from both the first and second capture sequences (A and B). Theoretically, using a 31 base strand, 31 to the 4^{th} power different capture sequences can be utilized, although in practice less will be available due to the desire to keep each subsequent capture sequence as distinct as possible from all of the capture sequences which preceeded it.

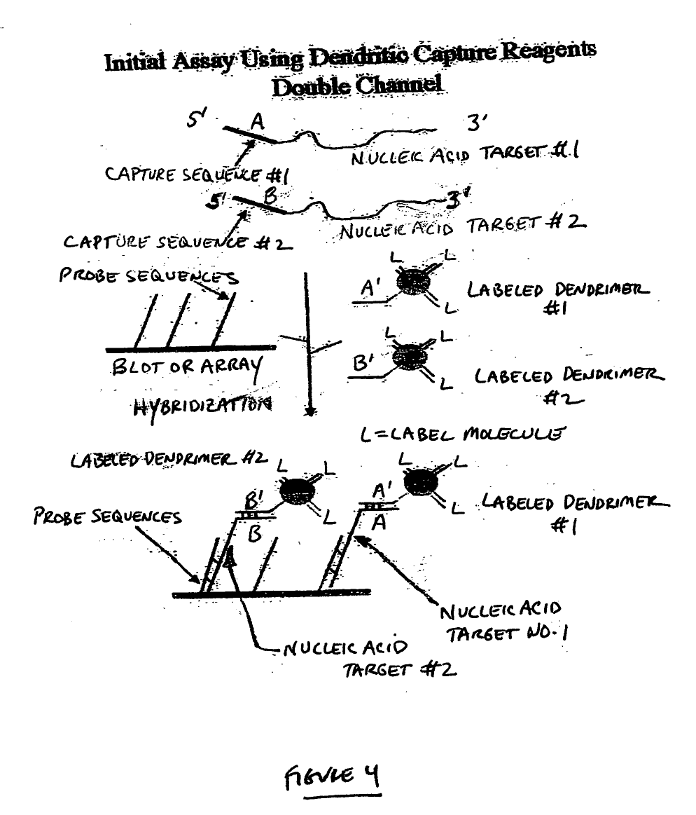

Similarly, the process can be used in the same manner for a dual channel assay as shown in FIGS. 4-6. The assay of FIG. 4 is conducted like that of FIG. 1, except that it is “dual channel”, i.e. designed to utilized two two different target sequences (each with its own unique capture sequence), as opposed to the assay of FIG. 1 which uses a single capture sequence. Likewise, three or more channels can also be provided, merely by using a new capture sequence for each channel.

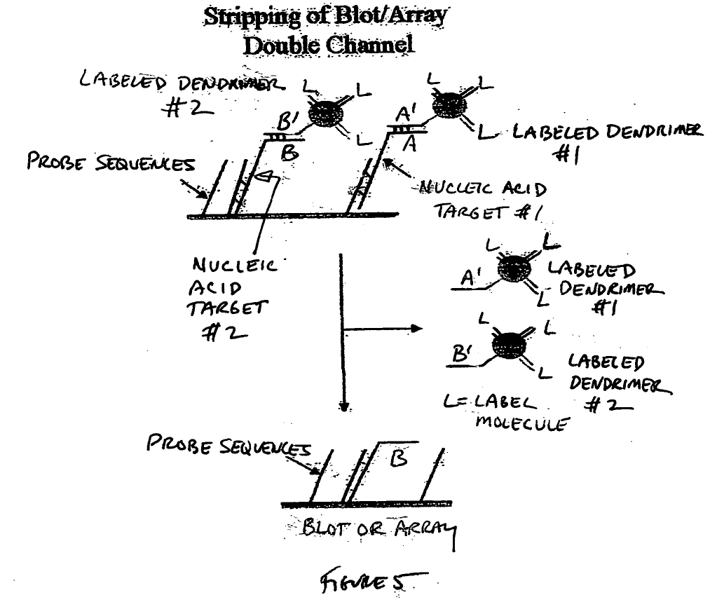

Following the initial assay of FIG. 4 (step one), the assay format is washed as shown in FIG. 5 (step two) to remove labelled dendrimers, as previously discussed with respect to FIG. 2. Once detection (step three) verifies that all label is gone, the microarray or blot can be reused in a further assay. For the purposes of illustration, a further dual channel assay is shown. However, this further assay can use as many channels as capture sequences are provided, whether single channel, dual channel, triple channel, or so forth.

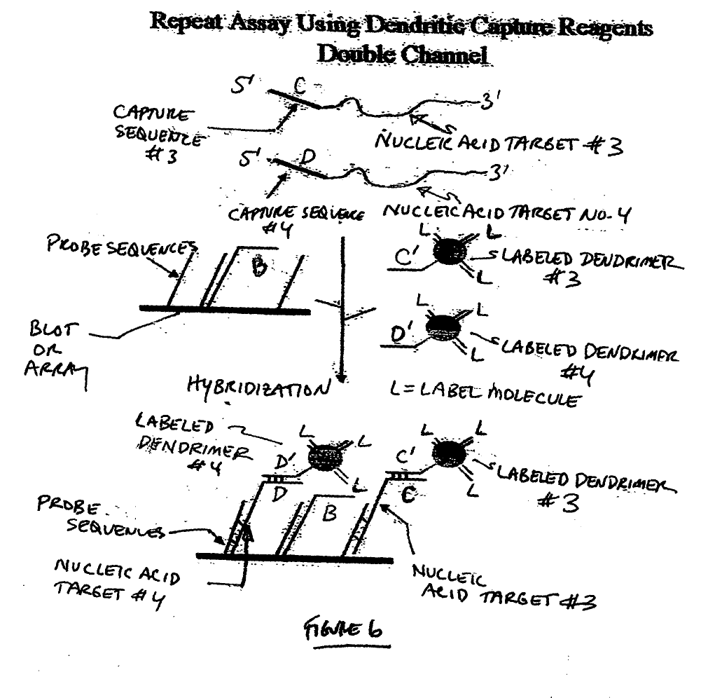

In step four of the invention, a new assay is conducted as shown in FIG. 6. In this second experiment, two new sets of target molecules and two new sets of dendrimers are used. This second experiment is prepared and conducted in similar fashion to the previous experiment of step one, and is conducted on the same microarray or blot surface that was used for the first experiment. This second assay uses two new capture sequences, sequences C and D (and their complements C′ and D′ on the dendrimer). These new capture sequences C and D are distinct from each other, and each is also distinct from the capture sequences A and B used in the experiment of FIG. 4.

If desired, the process can be repeated with further experiments, whether single channel, or dual channel, or triple or so forth.

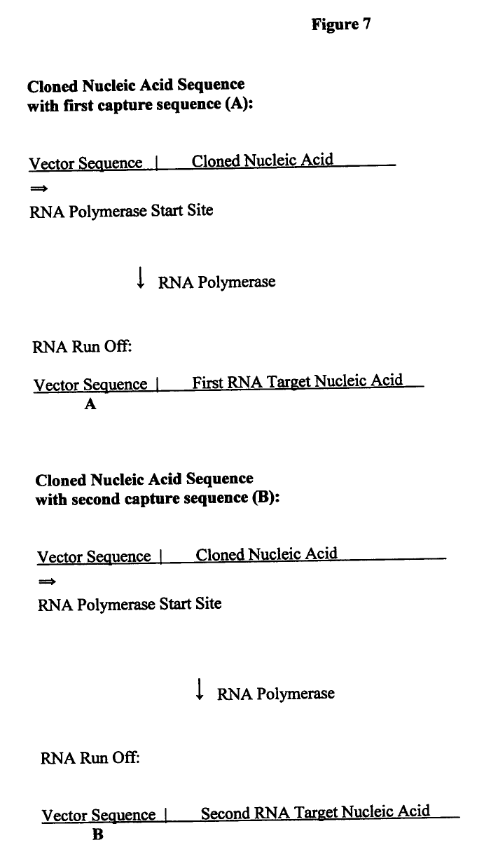

The target nucleic acid may be obtained from any desired source. For example, in accordance with one embodiment of the invention, as shown in FIG. 7, a vector is provided containing a cloned DNA fragment that will be used as the source for a target nucleic acid of RNA. The vector is linearized using restriction enzymes and RNA run offs are prepared using T₇, T₃ or SP₆ RNA polymerase, all using methods well known in the art.

The RNA transcribed from the cloned fragment by the polymerase will then be used as the target nucleic acid in the desired assays, such as the assays of FIGS. 1-6. The adjacent sequence of RNA transcribed from the vector sequence will be used as a capture sequence for the capture reagent. The capture reagent can be prepared by independently attaching oligonucleotides to the arms of the dendrimers which are complementary to the capture sequences.

Once the initial assays are conducted (single channel, dual channel, or so forth), a stripping step and signal detection step can be performed to prepare the arrays for reuse. The assay format can then be reused in a new assay by using new capture sequence, as discussed above. The cycle (assay, stripping, detection, new assay) can be repeated as often as desired, merely by using new capture sequences as discussed above.

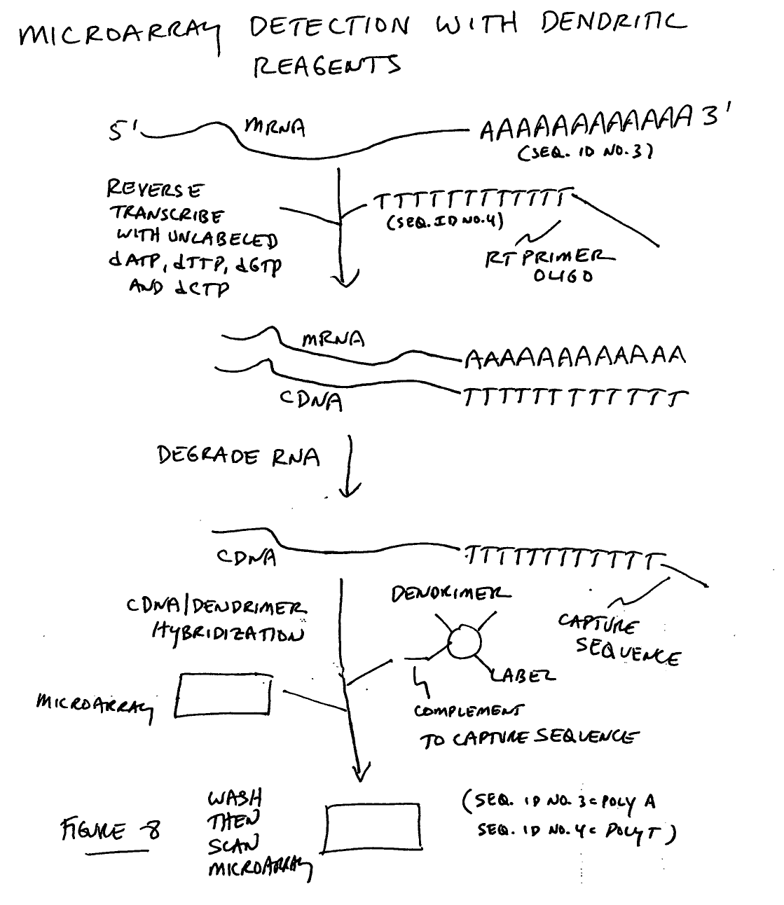

In a further embodiment, the target nucleic acid is in the form of a cDNA prepared from a biological sample, as shown in FIG. 8. The embodiment of FIG. 8, for example, can be used for RNA expression analysis using flourescent dendrimer based microarrays.

As previously discussed, the flourescent dendrimers are prepared by attaching two oligonucleotides to the outer surface arms of the core dendrimer structure (preferably 3DNA™). The first oligonucleotide is the complement to a capture nucleic acid sequence and will hybridized to and capture the 5 prime end of a reverse transcription primer, as discussed below. It can be attached by either ligation or hybridization followed by crosslinking. The second oligonucleotide is the label oligonucleotide which has a fluorescent dye molecule attached to either the 3′ end, 5′ end, both ends, or one or more internal nucleotide bases. The fluorescent oligonucleotide is hybridized and crosslinked to the complementary dendrimer binding arm. Any fluorescent dye that can be coupled to DNA can be attached to the dendrimers for this application. Examples include Cy3(™), Cy5(™), Fluorescent, Oregon Green(™), the Alexa(™) series dyes, and the BODIPY(™) series dyes to name a few. Each 3DNA reagent is labeled with at least 100 individual fluorescent molecules of the same type. The capture complement sequence is also designed to avoid any crosshybridization with the 3DNA core reagents and other published nucleic acid sequences, such as those found in public domain databases.

In this embodiment, the target nucleic acid reagent for use in determining the genomic information of a sample is often prepared from a RNA that is derived from a naturally occurring source. The RNA may be selected from total RNA, poly(A)+ RNA, amplified RNA and the like. If poly(A)+ RNA, the RNA can be part of the total cellular RNA or purified by published protocols or available kits.

For example, the initial RNA source may be present in a variety of different samples, and can be derived from a physiological source. The physiological source may be derived from a variety of eukaryotic sources, with physiological sources of interest including sources derived from single celled organisms such as yeast and multi-cellular organisms, including plants and animals, particularly mammals, where the physiological sources from multi-cellular organisms may be derived from particular organs or tissues of the multi-cellular organism, or from isolated cells derived therefrom.

In obtaining the RNA for processing and analysis, the physiological source may be subjected to a number of different processing steps, where such known processing steps may include tissue homogenation, cell isolation and cytoplasmic extraction, nucleic acid extraction and the like. Methods of isolating RNA from cells, tissues, organs or whole organisms are known to those of ordinary skill in the art and are described, for example, in Maniatis et al., Molecular Cloning: A Laboratory Manual, 2nd ed., Cold Spring Harbor Laboratory Press, 1989, and in Ausubel et al., Current Protocols in Molecular Biology, John Wiley & Sons, Inc., 1998, all of which are fully incorporated herein by reference.

Preferably, the extracted RNA is a polyadenylated RNA (poly(A)+ RNA). The poly(A)+ RNA includes an oligonucleotide which is comprised of a strand of adenine bases, or poly dA sequence, and provides a hybridization site for reverse transcription primers having a complementary oligonucleotide which is comprised of a strand of thymine bases, or poly dT sequence. This facilitates the attachment of the reverse transcription primers at appropriate sites to initiate the process of reverse transcription for forming the target nucleic acid reagent (e.g., cDNA) under suitable conditions.

It is noted that poly(A)+ RNA is typically present in most genomic samples and in all genomic samples of mammalian origin such as from humans, mice, rats, pigs and the like. However, the present invention may also be used in conjunction with non-poly(A)+ RNA samples as well. Such non-poly(A)+ RNA lacks a poly dA sequence useful as an attachment site for the RT primers. Accordingly, such non-poly(A)+ RNA is prepared by attaching or ligating a suitable attachment polynucleotide complementary to the RT primers used for facilitating reverse transcription.

The RT primer is a bifunctional oligonucleotide. It is composed of a 3′ oligo poly (dT) sequence and a 5′ dendrimer binding sequence (the dendrimer capture sequence). The 3′ oligo dT sequence serves as a primer for the RNA copying enzyme, reverse transcriptase, and can range in length from 15 to 30 nucleotides. This oligo dT sequence of the primer will hybridize to the complementary 3′ poly A tail of the mRNA and will serve as a starting point for the synthesis of DNA copies (cDNA) of the mRNA messages found in the sample. Reverse transcription is initiated in the presence of reverse transcriptase and deoxynucleotide triphosphates (i.e., dATP, dTTP, dGTP and dCTP). The mRNA is purged through suitable means including ethanol precipitation to yield the single stranded DNA or complementary DNA (cDNA), a target nucleic acid reagent. Reverse transcription from a population of total cellular RNA will yield a cDNA copy of the entire (poly A) population.

The polythymylated 5′ end of the cDNA inherits the capture sequence attached to the RT primer. The 5 prime dendrimer capture sequence, as the name implies, hybridizes to the complementary dendrimer sequence (preferably 3DNA), and bridges the fluorescent dendrimer to the cDNA.

In one preferred example, the RT primers are obtained from Genisphere, Inc. of Montvale, N.J. The nucleotide sequences of the primers corresponding to Cy3™ and Cy5™ are:

Cy3
5′-GGC CGA CTC ACT GCG CGT CTT CTG TCC CGC
C-oligo dT17-3′;
and
Cy5
5′-CCT GTT GCT CTA TTT CCC GTG CCG CTC CGG
T-oligo dT17-3′.

It is noted that these sequences can be found in Genisphere, Inc. protocols for their gene expression detection kits. The complement of the capture sequences are found on the fluor labeled capture reagents, or dendrimers. Although Cy3″ and Cy5″ are preferred embodiments, practically any fluor can be used, including, but not limited to, Alexa Fluors™ and other labeling dyes available from Molecular Probes, Inc. of Eugene, Oreg.

As diagrammed in FIG. 8, once the poly A RNA has been copied to form the cDNA molecules and the RNA has been degraded, a cDNA has been formed having the capture sequence attached thereto. This cDNA with capture sequence can be mixed with the corresponding dendrimer (preferably 3DNA reagent) and applied to the microarray by a typical hybridization reaction. The microarray includes probe nucleic acid affixed at specific locations on the array (the particular sequences of affixed nucleic acid probes also being referred to as the features of the array). Any cDNA molecules complementary to features on the array, will bind to that feature on the array and will remain immobile.

The dendrimer reagent in turn will hybridize to the 5′ end of the cDNA via the dendrimer capture sequence. Excess RT primer and unbound cDNA and dendrimer are then washed away. The array is scanned using the commercially available hardware and software to develop the signal.

Once the initial assay of FIG. 8 has been conducted (whether single channel, dual channel, or so forth), a stripping step and signal detection step can be performed to prepare the arrays for reuse. The assay format can then be reused for a new assay by using new capture sequence, as discussed above. The cycle (assay, stripping, detection, new assay) can be repeated as often as desired, merely by using new capture sequences as discussed above.

### Example 1:

With reference to FIG. 7 and generally to FIGS. 4-6, a method for nucleic acid detection using RNA Run-off probes and blot assays is as follows:

Preparation of RNA Run-Off Targets:

In vitro transcriptions reactions were prepared and run as described for the MAXIscript kit (Ambion, Austin, Tex.). Briefly, 250ng (2.0 μl) of plasmid p-Tri-Cyclin-D2 (Ambion, Austin, Tex.) was combined and mixed with 2 μL of 10× Transcription Buffer, 1 μl each of DATP, dCTP, dTTP and dGTP in a final volume of 17 μl in a 1.5 ml microfuge tube. One microliter (1.0 μl) of RNase Inhibitor was added to prevent the RNA product from degrading after synthesis. T7 RNA polymerase (2.0 μl) was added and the tube was mixed and briefly microfuged. The reaction mixture was incubated at 37° C. for 45 minutes to produce the RNA Run-off product (target). The reaction was terminated by heating to 65-70° C. for 15 minutes. The DNA template was removed by digesting by adding 1.0 ul of RNase-free DNase I and incubating the mixture at 37° C. for 15 minutes. The DNase digestion was stopped by adding 1 ul of 0.5M EDTA, pH=8.0. The RNA Run-off target was gel purified using a 10% TBE-Urea gel (Invitrogen, Carlsbad, Calif.) and eluting the probe into 1.5 mls of RNase free 50 mM Tris-HCl, 10 mM EDTA, pH=8.0. The target was stored at −70° C. until use. This RNA Run-off target contained DNA sequence corresponding to the Cyclin D2 gene as well as a short sequence (approximately 50 bases) that was derived from the DNA sequence of the plasmid located between the RNA polymerase start site and the cloned Cyclin D2 gene sequence Additional Run-off targets were prepared as described above using p-Tri-GAPDH, p-Tri Beta-Actin, and p-Tri-p53 (Ambion, Austin, Tex.) and purified and stored separately.

Preparation of 3DNA″ Dendrimers:

Cyclin D2, GAPDH Beta-Actin, and p53 capture dendrimer reagents were prepared by ligating an oligonucleotide that is complementary to the short sequence of nucleic acid between the RNA start site and the cloned gene sequence of the RNA Run-off targets to DNA dendrimer reagents by standard methods. These dendrimer attached oligonucleotide sequences, when mixed with the appropriate RNA Run-off, will hybridize with the complementary sequence on the RNA Run-off and link it to the 3DNA dendrimer reagent. The capture sequences for each RNA run-off were unique to each Run-off target to avoid cross-reactivity of one with that of the other.

Southern Blot Assay:

Initial Hybridization with Reference to FIG. 4:

A Southern blot was prepared using standard methods using dilutions of EcoRI restricted Human Genomic DNA. Briefly, samples of restricted genomic DNA (blot probes) equal to 5 μg, 1 ug, 0.2 μg, and 0.04 μg were separated by size on a 1% agarose gel and is subsequently transferred (blotted) using the standard method to a 6 cm by 20 cm piece of Hybond-N membrane (Amersham Pharmacia Biotech, Piscataway, N.J.). The genomic DNA was fixed to the membrane by UV cross-linking and the membrane (Southern blot) was transferred into a hybridization bag. Ten milliliters (10 mls) of ExpressHyb″ (Clontech, Palo Alto, Calif.) was added to the hybridization bag. The hybridization bag was sealed mixed and transferred into a 65° C. water bath for 30 minutes to prehybridize the membrane (the membrane prehybridization step).

32P (kinase) labeling of Dendrimer Label Oligonucleotides:

In a microfuge 1 μg (5 μl) of each of the oligonucleotides that bind to the free single stranded arms of dendrimer reagents was combined with 10 ul of 10× kinase buffer, 100 μCi of gamma 32P ATP (NEN, Boston, Mass.), 1 μl of T4 polynucleotide kinase (Amersham Pharmacia Biotech, Piscataway, N.J.) in a final volume of 100ul. The contents were mixed and incubated at 37° C. for 1 hour. The reaction was stopped by adding 2 ul of 0.5M EDTA, pH=8.0. The free unincorporated label nucleotide was removed by G-50 chromatography using Quick Spin Columns (Roche, Indianapolis, Ind.).

During the prehybridization 100 μl each of the gel purified Cyclin D2 and GAPDH RNA Run-off targets (1/15^{th}) were combined with 10 μl (200 ng) of the corresponding capture dendrimer in 0.5 mls of ExpressHyb′ and 10 μl of 32P labeled oligonucleotides. At the end of the 30 minutes, membrane prehybridization step, this mixture was added to the hybridization bag containing the Southern blot membrane. The Southern blot was hybridized overnight (˜16 hours) at 65° C. On the following morning, the hybridization bag containing the Southern blot was cut open and the membrane was transferred into 500 mls of 2×SSC, 1%SDS prewarmed to 65° C., and washed for 30 minutes. The membrane was transferred into prewarmed 2×SSC, 1%SDS and washed 30 minutes at 65° C. This wash step was repeated. The membranes were transferred into 0.5×SSC, 0.1%SDS and washed at 65° C. for 30 minutes. This wash step was repeated. The membrane was then drained of excess wash buffer and wrapped in plastic wrap, exposed to a Phosphor Screen and read using a STORM instrument (Molecular Dynamics, Sunnyvale, Calif.). A band of radioactive signal was observed at the position on the membrane corresponding to the Cyclin D2 and GAPDH genes.

Stripping of the Blot with Reference to FIG. 5:

The blot was removed from its wrapping and transferred into a glass tray containing 1 liter of 0.05×SSC/0.2% SDS in reagent grade deionized distilled water. Up to 4 blots per tray can be stripped at one time. The glass tray was placed into Reciprocal shaking water bath. The blot was washed for 40 minutes at 80° C. with constant shaking. The blot was wrapped in plastic wrap and exposed to a Phosphor Screen and read using a STORM instrument (Molecular Dynamics, Sunnyvale, Calif.) to confirm complete stripping of the blot. The blot was transferred into a hybridization bag.

Reprobing (Hybridization) the Blot with Reference to FIG. 6:

Ten milliliters (10 mls) of ExpressHyb″ (Clontech, Palo Alto, Calif.) was added to the hybridization bag. The hybridization bag was sealed mixed and transferred into a 65° C. water bath for 30 minutes to prehybridize the membrane. During the prehybridization 100 μl each of the gel purified Beta Actin and p53 RNA Run-off targets (1/15^{th}) were combined with 10μl (200 ng) of the corresponding capture dendrimer in 0.5 mls of ExpressHyb″ and 10 μl of 32P labeled oligonucleotides. At the end of the 30 minute membrane prehybridization step, this mixture was added to the hybridization bag containing the Southern blot membrane. The Southern blot was hybridized overnight (˜16 hours) at 65° C. On the following morning, the hybridization bag containing the Southern blot was cut open and the membrane was transferred into 500 mls of 2×SSC, 1%SDS prewarmed to 65° C. and washed for 30 minutes. The membrane was transferred into prewarmed 2×SSC, 1%SDS and washed 30 minutes at 65° C. This wash step was repeated. The membranes were transferred into 0.5×SSC, 0.1%SDS and washed at 65° C. for 30 minutes. This wash step was repeated. The membrane was then drained of excess wash buffer and wrapped in plastic wrap, exposed to a Phosphor Screen and read using a STORM instrument (Molecular Dynamics, Sunnyvale, Calif.). A band of radioactive signal was observed at the position on the membrane corresponding to the Beta Actin and p53 genes.

### EXAMPLE 2

With reference to FIG. 8 and generally to FIGS. 4-6, a method for detection and assay on a microarray is described below.

### Microarray Preparation

A microarray was prepared as directed by the manufacturer or by customary procedure protocol. The nucleic acid sequences comprising the DNA or gene probes were amplified using known techniques in polymerase chain reaction, then spotted onto glass slides, and processed according to conventional procedures.

Preparation and Concentration of Target Nucleic Acid Sequences Sample, or CDNA for Initial Hybridization with Reference to FIG. 4:

The target nucleic acid sequences, or cDNA was prepared from total RNA or poly(A)+RNA extracted from a sample of cells. It is noted that for samples containing about 10 to 20 μg of total RNA or 500-1000 ng of poly(A)⁺ RNA, ethanol precipitation is not required and may be skipped, because the cDNA is sufficiently concentrated to perform the microarray hybridization. In a microfuge tube, 0.25 to 5 μg of total RNA or 12.5 to 500 ng of poly(A)⁺ RNA was added with 3 μL of Cy3® (1)or Cy5® RT (1) primer (0.2 pmole) and RNase free water for a total volume of 10 μL to yield a RNA-RT primer mixture. The designation (1) after each primer refers to the specific capture sequence for the initial hybridization. The resulting mixture was mixed and microfuged briefly to collect contents in the bottom of the microfuge tube. The collected contents was then heated to 80 degrees C for about ten (10) minutes and immediately transferred to ice. In a separate microfuge tube on ice, 4 μL of 5× RT buffer, 1 μL of dNTP mix, 4 μL RNase free water, and 1 μL of reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture, was mixed briefly and incubated at 42° C. for two hours. The reaction was terminated by adding 3.5 μL of 0.5 M NaOH/50 mM EDTA to the mixture. The mixture was incubated at 65° C. for ten (10) minutes to denature the DNA/RNA hybrids and the reaction was neutralized with 5 μL of 1 M Tris-HCl, pH 7.5. 38.5 μL of 10 mM Tris, pH 8.0, 1 mM EDTA was then added to the neutralized reaction mixture. (The above steps may be repeated replacing the 3 μL of Cy3® RT primer (0.2 pmole) with 3 μL of Cy5® RT primer (0.2 pmole) for preparing dual channel expression assays whereby the prepared Cy3® and Cy5® cDNA mixture are mixed together with 10 μL of 10 Tris, pH 8.0, 1 mM EDTA, to yield a reaction mixture for processing in the following steps.)

2 μL of a carrier nucleic acid (10 mg/mL linear acrylamide) was added to the neutralized reaction mixture for ethanol precipitation. 175 μL of 3M ammonium acetate was added to the mixture and then mixed. Then, 625 μL of 100% ethanol was added to the resulting mixture. The resulting mixture was incubated at −20 degrees C for thirty (30) minutes. The sample was centrifuged at an acceleration rate greater than 10,000 g for fifteen (15) minutes. The supernatant was aspirated and then 330 μL of 70% ethanol was added to the supernatant, or cDNA pellet. The cDNA pellet was then centrifuged at an acceleration rate greater than 10,000 g for 5 minutes, and was then removed. The cDNA pellet was dried (i.e., 20-30 minutes at 65° Celsius).

Hybridization of CDNA/Dendrimer Probe Mixture to Microarray

The DNA hybridization buffer was thawed and resuspended by heating to 65° C. for ten (10) minutes. The hybridization buffer comprised of 40% formamide, 4×SSC, and 1%SDS. The buffer was mixed by inversion to ensure that the components were resuspended evenly. The heating and mixing was repeated until all of the material was resuspended. A quantity of competitor DNA was added as required (e.g. 1 μg COT-1-DNA, and 0.5 μg polydT). The cDNA was resuspended in 5.0 μL of sterile water.

In a first embodiment, single channel analysis, 2.5 μL of one type of 3DNA® reagent (Genisphere, Inc., Montvale, N.J.) (Cy3 or Cy5) with the appropriate capture sequence was added to the resuspended cDNA along with 12.5 μL of a DNA hybridization buffer (containing 40% formamide). In an alternative embodiment, for dual channel analysis, 2.5 μL of two types of 3DNA® reagents, Cy3 and Cy5 specifically labeled dendrimers, with the appropriate capture sequences were added to the resuspended cDNA along with 10 μl of a DNA hybridization buffer. In a further embodiment of multiple channel analysis (with three or more channels), 2.5 μL of three or more types of 3DNA® reagents, Cy3, Cy5, and one or more prepared using another label moiety, with the appropriate capture sequences were added to the resuspended cDNA along with 10 μL of a DNA hybridization buffer.

For larger hybridization buffer volumes, additional DNA hybridization buffer may be added to the required final volume. It is noted that hybridization buffer volumes greater than 35 μL may also require additional 3DNA® reagents.

The DNA hybridization buffer mixture was incubated at about 45-50° C. for about 15 to 20 minutes to allow for prehybridization of the capture sequence on the cDNA to the complementary sequence on the 3DNA® reagents. The prehybridized mixture was then added to the microarray and then incubated overnight at 55° C. At this stage the cDNA was hybridized to the gene probes.

Post Hybridization Wash:

The microarray was briefly washed to remove any excess dendrimer probes. First, the microarray was washed for 10 minutes at 55° C. with 2×SSC buffer, 0.2%SDS. Then the microarray was washed for 10 minutes at room temperature with 2×SSC buffer. Finally the microarray was washed for 10 minutes at room temperature with 0.2×SSC buffer.

Signal Detection:

The microarray was then scanned as directed by the scanner's manufacturer for detecting, analyzing, and assaying the hybridization pattern.

Microarray Stripping Procedure with reference to FIG. 5:

The microarray was incubated in 0.1 M NaOH for 20-30 minutes at 50° C. with agitation to remove the hybridized 3DNA reagents from the bound target (as illustrated in FIG. 2). The array was transferred into deionized distilled water for 2 minutes and then 2×SSC for 2 minutes. The array was transferred into 0.2×SSC for 2 minutes and finally the excess buffer removed by centrifugation in a 50 ml centrifuge tube at 1000 rpm for 2 minutes. The array was scanned to confirm that all signal was removed.

Preparation and Concentration of Target Nucleic Acid Sequences Sample, or CDNA for Second Hybridization with Reference to FIG. 6:

A second target nucleic acid, or cDNA, was prepared from total RNA or poly(A)+RNA extracted from a sample of cells. It is noted that for samples containing about 10 to 20 μg of total RNA or 500-1000 ng of poly(A)⁺ RNA, ethanol precipitation is not required and may be skipped, because the cDNA is sufficiently concentrated to perform the microarray hybridization. In a microfuge tube, 0.25 to 5 μg of total RNA or 12.5 to 500 ng of poly(A)⁺ RNA was added with 3 μL of Cy3® (2)or Cy5® (2) RT primer (0.2 pmole) and RNase free water for a total volume of 10 μL to yield a RNA-RT primer mixture. The designation (2) after each primer refers to the specific capture sequence for the second hybridization. The labels, Cy3 and Cy5 are the same as that of the initial hybridization but the capture sequences are different for each primer and capture reagent for subsequent rehybridizations. The resulting mixture was mixed and microfuged briefly to collect contents in the bottom of the microfuge tube. The collected contents was then heated to 80° C. for about ten (10) minutes and immediately transferred to ice. In a separate microfuge tube on ice, 4 μL of 5×RT buffer, 1 μL of dNTP mix, 4 μL RNase free water, and 1 μL of reverse transcriptase enzyme (200 Units) were combined to yield a reaction mixture. The reaction mixture was gently mixed and microfuged briefly to collect contents in the bottom of the microfuge tube. 10 μL of the RNA-RT primer mixture and 10 μL of the reaction mixture, was mixed briefly and incubated at 42° C. for two hours. The reaction was terminated by adding 3.5 μL of 0.5 M NaOH/50 mM EDTA to the mixture. The mixture was incubated at 65 ° C. for ten (10) minutes to denature the DNA/RNA hybrids and the reaction was neutralized with 5 μL of 1 M Tris-HCl, pH 7.5. 38.5 μL of 10 mM Tris, pH 8.0, 1 mM EDTA was then added to the neutralized reaction mixture. (The above steps may be repeated replacing the 3 μL of Cy3® RT primer (0.2 pmole) with 3 μL of Cy5® RT primer (0.2 pmole) for preparing dual channel expression assays whereby the prepared Cy3® and Cy5® cDNA mixture are mixed together with 10 μL of 10 Tris, pH 8.0, 1 mM EDTA, to yield a reaction mixture for processing in the following steps.)

2 μL of a carrier nucleic acid (10 mg/mL linear acrylamide) was added to the neutralized reaction mixture for ethanol precipitation. 175 μL of 3M ammonium acetate was added to the mixture and then mixed. Then, 625 μL of 100% ethanol was added to the resulting mixture. The resulting mixture was incubated at −20 degrees C for thirty (30) minutes. The sample was centrifuged at an acceleration rate greater than 10,000 g for fifteen (15) minutes. The supernatant was aspirated and then 330 μL of 70% ethanol was added to the supernatant, or cDNA pellet. The cDNA pellet was then centrifuged at an acceleration rate greater than 10,000 g for 5 minutes, and was then removed. The cDNA pellet was dried (i.e., 20-30 minutes at 65° Celsius).

### Hybridization of CDNA/Dendrimer Probe Mixture to Microarray

The DNA hybridization buffer was thawed and resuspended by heating to 65° C. for ten (10) minutes. The hybridization buffer comprised of 40% formamide, 4×SSC, and 1%SDS. The buffer was mixed by inversion to ensure that the components were resuspended evenly. The heating and mixing was repeated until all of the material was resuspended. A quantity of competitor DNA was added as required (e.g. 1 μg COT-1-DNA, and 0.5 μg polydT). The cDNA was resuspended in 5.0 μL of sterile water.

In a first embodiment, single channel analysis, 2.5 μL of one type of 3DNA® reagent (Genisphere, Inc., Montvale, N.J.) (Cy3 or Cy5) with the appropriate capture sequence was added to the resuspended cDNA along with 12.5 μL of a DNA hybridization buffer (containing 40% formamide). In an alternative embodiment, for dual channel analysis, 2.5 μL of two types of 3DNA® reagents, Cy3 and Cy5 specifically labeled dendrimers, with the appropriate capture sequences were added to the resuspended cDNA along with 10 μl of a DNA hybridization buffer. In a further embodiment of multiple channel analysis (with three or more channels), 2.5 μL of three or more types of 3DNA® reagents, Cy3, Cy5, and one or more prepared using another label moiety, with the appropriate capture sequences were added to the resuspended cDNA along with 10 μL of a DNA hybridization buffer.

For larger hybridization buffer volumes, additional DNA hybridization buffer may be added to the required final volume. It is noted that hybridization buffer volumes greater than 35 μL may also require additional 3DNA® reagents.

The DNA hybridization buffer mixture was incubated at about 45-50° C. for about 15 to 20 minutes to allow for prehybridization of the capture sequence on the cDNA to the complementary sequence on the 3DNA® reagents. The prehybridized mixture was then added to the microarray and then incubated overnight at 55 ° C. At this stage the cDNA was hybridized to the gene probes.

### Post Hybridization Wash

The microarray was briefly washed to remove any excess dendrimer probes. First, the microarray was washed for 10 minutes at 55° C. with 2×SSC buffer, 0.2%SDS. Then the microarray was washed for 10 minutes at room temperature with 2×SSC buffer. Finally the microarray was washed for 10 minutes at room temperature with 0.2×SSC buffer.

### Signal Detection

The microarray was then scanned as directed by the scanner's manufacturer for detecting, analyzing, and assaying the hybridization pattern.

The foregoing discussion and examples disclose and describe merely exemplary embodiments of the present invention. One skilled in the art will readily recognize from such discussion, and from the accompanying claims, that various changes, modifications, and variations can be made therein without departing from the spirit and scope of the invention as defined in the following claims.

1-22. (cancelled)

23. A method for reusing an assay, comprising the steps of:
(a) conducting a first assay, said first assay comprising:
(i) a first hybridization of a target nucleic acid to probe nucleic acid located on an assay format, and
(ii) hybridization of a first dendrimer to said target nucleic acid, wherein said target nucleic acid comprises a first capture sequence which hybridizes with a complementary nucleic acid sequence of said first dendrimer;
(b) stripping said first dendrimer from said target nucleic acid; and,
(c) conducting a second assay on said assay format, said second assay comprising:
(i) a second hybridization of target nucleic acid to probe nucleic acid on the same assay format used for said first assay; and,
(ii) hybridization of a second dendrimer to the target nucleic acid of said second assay, wherein said target nucleic acid of said second assay comprises a second capture sequence for hybridization to said second dendrimer, said second capture sequence being a nucleic acid sequence which is different from the nucleic acid sequence of said first capture sequence.

24. A method as claimed in claim 23, wherein said first dendrimer comprises a label for producing a detectable signal.

25. A method as claimed in claim 23, wherein said second dendrimer comprises a label for producing a detectable signal.

26. A method as claimed in claim 24, wherein said label is a flourescent label.

27. A method as claimed in claim 25, wherein said label is a flourescent label.

28. A method as claimed in claim 24, further comprising the step of detecting said signal of said label of said first dendrimer before said stripping of said dendrimer from said target nucleic acid.

29. A method as claimed in claim 25, further comprising the step of detecting said signal of said label of said second dendrimer.

30. A method as claimed in claim 24, wherein said stripping of said first dendrimer is followed by a detection of any of said label on said assay format to verify that none of said label of said first dendrimer can be detected on said assay format.

31. A method as claimed in claim 23, wherein said assay format is a blot.

32. A method as claimed in claim 23, wherein said assay format is a microarray.

33. A method as claimed in claim 23, wherein at least one of said first and second assays comprises single channel detection.

34. A method as claimed in claim 23, wherein at least one of said first and second assays comprises dual channel detection.

35. A method as claimed in claim 23, further comprising the step of conducting a third assay on said format using a target nucleic acid comprising a third capture sequence, said third capture sequence comprising a nucleic acid sequence which is different from the nucleic acid sequences of both said first capture sequence and said second capture sequence.

36. A method as claimed in claim 23, further comprising the step of conducting a third assay on said format using a target nucleic acid comprising a third capture sequence, said third capture sequence comprising a nucleic acid sequence which is different from the nucleic acid sequences of both said first capture sequence and said second capture sequence.

37. A method as claimed in claim 23, further comprising the step of conducting further assays on said format using target nucleic acids comprising capture sequences which are different from the capture sequences used in any of the prior assays on said assay format.

38. A method as claimed in claim 23, wherein said capture sequence comprises 31 base pairs.

39. A method as claimed in claim 23, wherein at least one of said first and second assays is used for RNA expression analysis.
